(19) United States
(12) Patent Application Publication
(10) Pub. No.: US 2016/0213331 A1
(43) Pub. Date: Jul. 28, 2016

(54) MOBILE TERMINAL HAVING FUNCTIONS OF MEASURING BIOMETRIC SIGNALS AND ESTIMATING AND MONITORING BLOOD PRESSURE IN REAL TIME BASED ON MEASURED BIOMETRIC SIGNALS

(71) Applicant: HUINNO, CO., LTD., Seongnam (KR)

(72) Inventors: Yeong Joon GIL, Busan (KR); Min Yong SHIN, Suwon (KR)

(21) Appl. No.: 14/864,262

(22) Filed: Sep. 24, 2015

(30) Foreign Application Priority Data
Jan. 27, 2015 (KR) 10-2015-0012855
Mar. 6, 2015 (KR) 10-2015-0031979
Mar. 6, 2015 (KR) 10-2015-0031980
Mar. 9, 2015 (KR) 10-2015-0032676
Apr. 10, 2015 (KR) 10-2015-0051015

(51) Int. Cl. A61B 5/00; A61B 5/1455; A61B 5/0408; A61B 5/0205; A61B 5/024

(52) U.S. Cl. CPC A61B 5/7278; A61B 5/0205; A61B 5/02433; A61B 5/04085; A61B 5/14551; A61B 5/6898; A61B 5/742; A61B 5/021

(57) ABSTRACT

A mobile terminal having a function of measuring biometric signals from a user is provided. The mobile terminal includes a main body and a display, which is formed at a front of the main body and configured to display information to a user. The display includes a measurement area configured to measure biometric signals from the user. A pixel structure formed in the measurement area of the display includes red (R) sub-pixels configured to generate R light, and infrared (IR) sub-pixels configured to generate IR light. The measurement area of the display includes a light-receiver, which receives light reflected from the body of the user. The mobile terminal is configured to measure at least one of a photoplethysmogram (PPG) signal and an oxygen saturation (SpO₂) signal through the measurement area.

### CROSS-REFERENCE TO RELATED APPLICATIONS

This application claims priority from

1. Korean Patent Application No. 10-2015-0032676 filed on Mar. 9, 2015,

2. Korean Patent Application No. 10-2015-0012855 filed on Jan. 27, 2015,

3. Korean Patent Application No. 10-2015-0051015 Apr. 10, 2015,

4. Korean Patent Application No. 10-2015-0031980 Mar. 6, 2015,

5. Korean Patent Application No. 10-2015-0031979 Mar. 6, 2015, in the Korean Intellectual Property Office. The application is incorporated by reference herein.

### TECHNICAL FIELD

The present disclosure relates to a mobile terminal having the functions of measuring various biometric signals from a user and estimating the blood pressure of the user in real time based on the biometric signals.

### BACKGROUND

Due to rapid developments in science and technology, the quality of life of the entire human race has been improved, bringing many changes to the medical environment. It can take a few hours or days to have medical images, such as X-ray, computerized tomography (CT) or functional magnetic resonance imaging (fMRI) images, interpreted. However, picture archive communication systems (PACS) have been introduced that capture medical images and transmit the captured medical images directly to the screen of a radiologist's monitor for rapid interpretation by the radiologist. Also, ubiquitous-Healthcare (u-Healthcare)-related medical appliances that enable patients to check their blood sugar and blood pressure anytime anywhere have been developed and widely distributed.

In particular, in the case of hypertension, which may be a major cause of various diseases and has an ever-increasing prevalence, systems are needed to regularly measure blood pressure and to report the results of the measurement in real time. A method may be used that can reduce the number of a patient's visits to a doctor using a u-Healthcare technique in which a blood pressure measuring sensor is inserted into the pulmonary artery of a patient with a chronic heart disease to measure the blood pressure of the patient and transmit the result of the measurement to a doctor, in real time, via radio communication. The doctor may then deliver a prescription to the patient while monitoring changes in the pulmonary blood pressure of the patient from a remote place. However, this conventional u-Healthcare method involves a rather invasive measurement process, even though it can continuously and precisely measure blood pressure, and may thus face many difficulties and risks (such as possible damage or inflammation to the arteries). Accordingly, the conventional u-Healthcare method has a limited use.

Thus a method capable of measuring blood pressure in real time in a non-invasive manner without the need to insert a blood pressure measuring sensor in the artery would be beneficial, but a user may also wish to correct his or her blood pressure through biofeedback of the blood pressure monitored and measured in a ubiquitous environment. A non-invasive blood pressure measurement method has been introduced in which a cuff is attached onto the arm of a user to measure the blood pressure. However, this non-invasive blood pressure measurement method requires either the user or somebody else to operate a blood pressure measuring device to measure blood pressure. Accordingly, it may be difficult to continuously measure blood pressure. Also, it may be a slow process, taking more than a few dozens of seconds to measure blood pressure.

Therefore, the introduction of a system capable of continuously measuring and monitoring blood pressure in a non-invasive manner so as to alert people to the danger of hypertension and thus to help them receive timely care in case of an emergency is needed.

### SUMMARY

The present disclosure relates to addressing at least the problems and disadvantages described above, and relates to providing at least the advantages described below. An exemplary embodiment of the present disclosure provides measuring various biometric signals (such as electrocardiogram (ECG), photoplethysmogram (PPG) and oxygen saturation (SpO₂) signals) from the body of a user.

Another exemplary embodiment of the present disclosure provides continuously monitoring blood pressure in a non-invasive manner in a ubiquitous environment by estimating the blood pressure of a user in real time based on measured biometric signals. Another exemplary embodiment of the present disclosure provides allowing biometric signals to be measured by mobile terminals (such as smartphones) that people carry around so as to help users to measure biometric signals with ease regardless of time and location However, embodiments of the present disclosure are not restricted to those set forth herein. The above and other embodiments of the present disclosure will become more apparent to one of ordinary skill in the art to which the present disclosure pertains by referencing the detailed description of the present disclosure given below.

According to an embodiment of the present disclosure, a mobile terminal having a function of measuring biometric signals can include a main body; and a display formed at a front of the main body and displaying information to a user, where the display includes a measurement area for measuring biometric signals from the user. A pixel structure formed in the measurement area of the display can include red (R) sub-pixels, which generate R light, and infrared (IR) sub-pixels, which generate IR light. the measurement area E of the display can include a light-receiver, which receives light reflected from the body of the user, and at least one of a PPG signal and a SpO₂ signal is measured through the measurement area.

The mobile terminal may also include a first electrode disposed on a display screen of the display and a second electrode formed in a portion of the main body where the display is not provided, where at least one of an ECG signal, the PPG signal and the SpO₂ signal is measured through the first and second electrodes and the measurement area. The second electrode B may be formed at one of a left side, a right side, the top, the bottom, or the rear of the main body. The mobile terminal may also include a third electrode provided at a location apart from the second electrode in the main body.

The mobile terminal may also include a processor analyzing measured biometric signals, where the processor is embedded in the main body. The processor may estimate blood pressure of the user in real time based on the measured biometric signals. The display may display at least one of information regarding measured biometric signals, results of analysis of the measured biometric signals, and the user's blood pressure estimated based on the measured biometric signals to the user. The display may be embedded in the main body. The mobile terminal may be implemented as a smartphone.

According to another exemplary embodiment of the present disclosure, a mobile terminal having a function of measuring biometric signals includes a main body; an ECG sensor including a first electrode that is formed in a home button provided in the main body; a second electrode that is formed in a portion of the main body where the home button is not provided; and a PPG sensor formed in a portion of the main body where the first or second electrode is formed and having a light-emitter that generates light to be irradiated to the body of a user and a light-receiver that receives light reflected from the body of the user. At least one of an ECG signal, a PPG signal, and a SpO₂ signal is measured using at least one of the ECG sensor and the PPG sensor.

The PPG sensor may be formed in the home button where the first electrode is formed. The second electrode may be formed at one of a left side, a right side, the top, the bottom and the rear of the main body. The ECG may also include a third electrode provided at a location apart from the second electrode in the main body. The mobile terminal may also include a processor analyzing biometric signals measured by the ECG sensor or the PPG sensor, where the processor is embedded in the main body. The processor may estimate blood pressure of the user in real time based on the biometric signals measured by the ECG sensor and/or the PPG sensor.

The mobile terminal may also include a display displaying at least one of information regarding biometric signals measured by the ECG sensor or the PPG sensor, results of analysis of the measured biometric signals, and the user's blood pressure estimated based on the measured biometric signals to the user, where the display is embedded in the main body. The light-emitter of the PPG sensor may include an R light-emitting diode (LED), which emits R light, and an IR LED, which emits IR light, and the light-receiver of the PPG sensor may include at least one of a photodiode and a phototransistor. According to various exemplary embodiments, various biometric signals that may be generated in the body of the user can be measured with a mobile terminal that the user carries around. Accordingly, the user can easily measure biometric signals regardless of time and location.

In addition, the blood pressure of the user can be estimated in real time based on biometric signals measured with a mobile terminal. Accordingly, it is possible to continuously monitor blood pressure in a non-invasive manner in a ubiquitous environment. Thus, hypertensive patients can be alerted to the danger of hypertension and hypertension-related disease and can properly manage their health, and even people with no apparent blood pressure problems can also benefit by having their blood pressure information monitored so as to prevent hypertension in advance. Moreover, measured biometric signals can be effectively analyzed by a microprocessor embedded in a mobile terminal such as a smartphone, and the measured and/or analyzed biometric signals can be readily provided to the user via the display of the mobile terminal.

Furthermore, biometric signals measured and/or analyzed by a mobile terminal can be transmitted to another device of the user or a remote server via a communication function of the mobile terminal. Accordingly, a considerable amount of information can be effectively stored and managed, and can be used by multiple users at the same time. Other features and embodiments will be apparent from the following detailed description, the drawings, and the claims.

### BRIEF DESCRIPTION OF THE DRAWINGS

FIG. 1 is a schematic block diagram illustrating an entire system according to an exemplary embodiment of the present disclosure;

FIG. 2 is a block diagram of a mobile terminal having the functions of measuring biometric signals and estimating blood pressure in real time based on the measured biometric signals according to an exemplary embodiment of the present disclosure;

FIGS. 3 to 6 are diagrams illustrating a first example of the mobile terminal;

FIG. 7 is a diagram illustrating a second example of the mobile terminal; and

FIGS. 8 and 9 are diagrams illustrating a third example of the mobile terminal.

### DETAILED DESCRIPTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although exemplary embodiments may be described as using a plurality of units to perform the exemplary process, it is understood that the exemplary processes may also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Embodiments of the present disclosure are described in detail below with reference to the accompanying drawings. The embodiments of the present disclosure are sufficiently described in detail such that those skilled in the art may carry out the present disclosure. It should be understood that although various embodiments of the present disclosure are different from each other, they need not be mutually exclusive. For example, in regard to an embodiment, specific forms, structures, and characteristics described herein can be realized through another embodiment without departing from the spirit and scope of the present disclosure. Moreover, it should be understood that locations or arrangements of separate elements within the disclosed embodiments can be changed without departing from the spirit and scope of the present disclosure. Accordingly, detailed descriptions which will be given below are not intended to be restrictive, and the scope of the present disclosure, if properly described, should be limited only by the accompanying claims and equivalents thereof. Similar reference numerals shown in the drawings denote elements performing identical or similar functions in several aspects.

Embodiments will hereinafter be described with reference to the accompanying drawings.

### Structure of Entire System

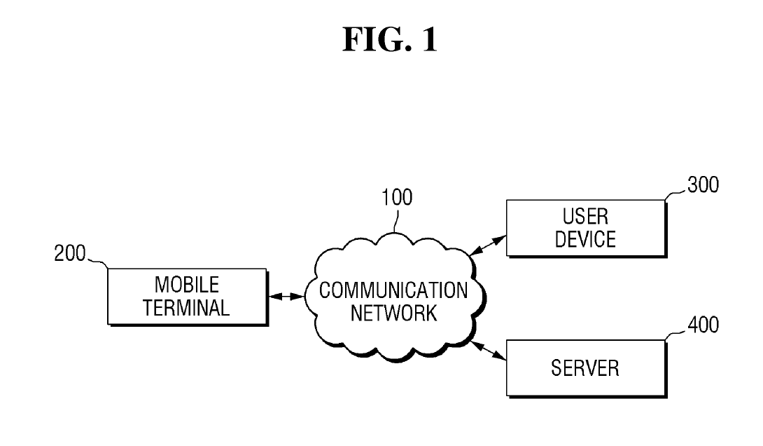

Referring to FIG. 1, the system may include a communication network 100, a mobile 200 being capable of measuring biometric signals and estimating blood pressure based on the measured biometric signals, a remote user device 300, and a remote server 400. The communication network 100 may not necessarily be limited to a particular communication method such as a wired or wireless communication method, and may be implemented as various communication networks such as a local area network (LAN), a metropolitan area network (MAN) or a wide area network (WAN). Examples of the communication network 100 may include LANs such as a Wireless-Fidelity (Wi-Fi) network, a Wi-Fi Direct network, a Long-Term Evolution (LTE) Direct network, and/or a Bluetooth network that are already well known, but the present disclosure is not limited thereto. That is, the communication network 100 may at least partially include a typical wired/wireless communication network, a typical telephone network, and/or a typical wired/wireless television communication network.

The mobile terminal 200 may be capable of measuring various biometric signals from a user with the use of sensors installed in the main body thereof and estimating the blood pressure of the user in real time based on the measured biometric signals. The term “mobile terminal”, as used herein, indicates a personal terminal capable of allowing a user to communicate with another user, access the Internet, and watch moving images or broadcasts, and encompasses a mobile phone, a personal digital assistant (PDA), a tablet personal computer (PC), etc. Units, modules, emitters, and/or receivers may be operated by a controller having a memory and a processor.

The mobile terminal 200 may include an electrocardiogram (ECG) sensor that has a plurality of electrodes formed apart from one another, and a photoplethysmogram (PPG) sensor that has a light-emitter and a light-receiver, and may be configured to measure various biometric signals (such as, for example, ECG, PPG and/or oxygen saturation (SpO₂) signals) from the body of a user with the use of the ECG and PPG sensors and estimate the blood pressure of the user in real time based on the measured biometric signals. Biometric signals measured by the sensors of the mobile terminal 200 may be analyzed by a processor of the mobile terminal 200. The analyzed biometric signals may be provided directly to the user via a display of the mobile terminal 200. Alternatively, the analyzed biometric signals may be transmitted to the remote user device 300 or the remote server 400 via the communication network 100 and may be stored and managed by the remote user device 300 or the remote server 400.

### Structure of Mobile Terminal

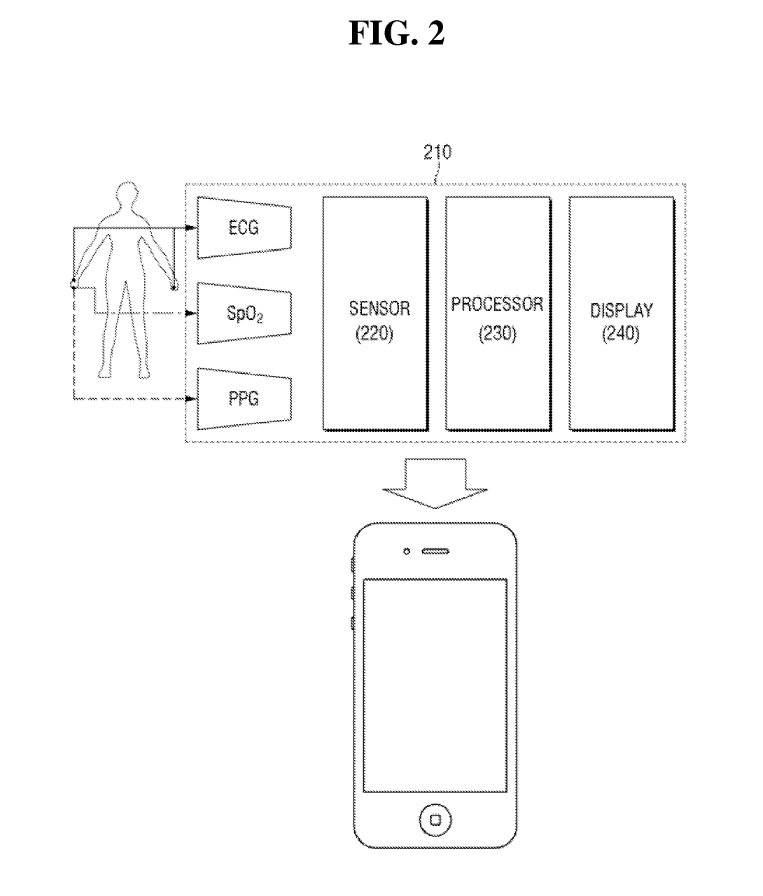

FIG. 2 shows a block diagram of a mobile terminal capable of measuring biometric signals and estimating blood pressure in real time based on the measured biometric signals, according to, e.g., the mobile terminal 200. Referring to FIG. 2, the mobile terminal 200 may include a main body 210, a sensor 220 that may measure various biometric signals from a user, a processor 230 that may analyze the biometric signals measured by the sensor 220, and a display 240 that may output the results of the measurement performed by the sensor 220 and/or the results of the analysis performed by the processor 230 to the user.

The sensor 220 may include an ECG sensor that may measure an ECG signal from the user with the use of a plurality of electrodes that are formed apart from one another and a PPG sensor (or an optical sensor) that may measure a PPG signal and/or an SpO₂ signal from the user with the use of a light-emitter and a light-receiver. By using the mobile terminal 200 equipped with the sensor 220 (i.e., the ECG sensor and/or the PPG sensor), it is possible to continuously measure various biometric signals (such as ECG, PPG and SpO₂ signals) from the human body in a non-invasive manner.

An ECG signal is a waveform consisting of the vector sum of action potentials produced by the special excitatory and conductive system of the heart. That is, an ECG signal is the vector sum of action potentials produced by various parts of the heart such as the sinoatrial node, the atrioventricular node, the His bundle, the bundle branches, and the Purkinje fibers and can be measured by electrodes attached to the outside of the human body. For example, an ECG signal may be obtained by a standard limb lead method. For example, an ECG signal may be measured by a plurality of electrodes formed in the main body 210 of the mobile terminal 200.

A PPG signal is a pulse wave signal measured from the peripheral blood vessels in response to blood ejected from the heart during ventricular systole being delivered to the peripheral blood vessels. A PPG signal may be measured by using the optical properties of biological tissues. For example, a PPG sensor (or an optical sensor), which is capable of measuring a pulse wave signal, may be attached to part of the human body such as the tip of a finger or toe where peripheral blood vessels are distributed. The PPG sensor may also measure and translate changes in the amount of light into changes in the amount of blood flow, which are changes in the volume of the peripheral blood vessels. For example, a PPG signal may be measured by irradiating red (R) light generated by the light-emitter of the PPG sensor in the main body 210 to the human body and observing changes in the amount of light received by the light-receiver of the PPG sensor after being reflected from the human body. A PPG signal may not necessarily be used alone. Instead, information such as pulse transit time (PTT) information or pulse wave velocity (PWV) information may be extracted by analyzing the correlation between a PPG signal and an ECG signal, and may be used to diagnose cardiovascular disease. For example, a feature point may be obtained from the second derivative of a PPG signal, the interval between the feature point and the peak of (an R wave of) an ECG signal may be measured so as to extract a PTT signal and a PWV signal, and the PTT signal and the PWV signal may be used to diagnose the state of blood vessels, arteriosclerosis, peripheral circulation disturbance, etc.

A SpO₂ signal is a biometric signal indicating the amount of oxygen present in blood, and particularly, in hemoglobin. As an example, a SpO₂ signal may be measured by alternately irradiating R light and infrared (IR) light to a peripheral blood vessel area at intervals of part of the human body where peripheral blood vessels are concentrated and observing changes in the amount of light received by a light-receiver after being reflected from the human body. For example, a SpO₂ signal may be measured by the PPG sensor (or the optical sensor).

Examples of the mobile terminal 200 being capable of measuring biometric signals and estimating blood pressure in real time based on the measured biometric signals will hereinafter be described.

### FIRST EXAMPLE OF MOBILE TERMINAL

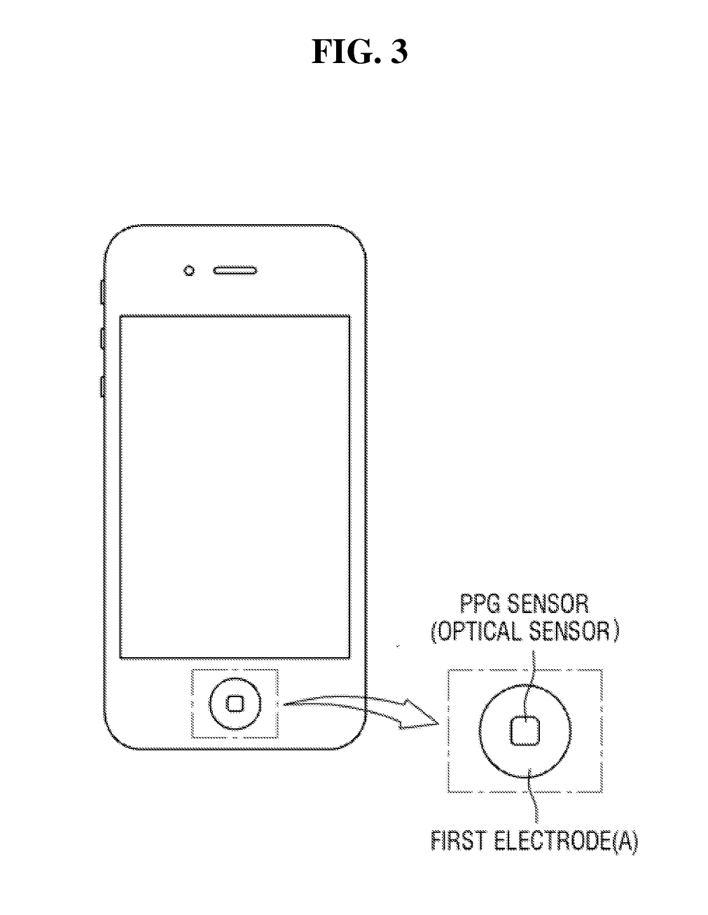

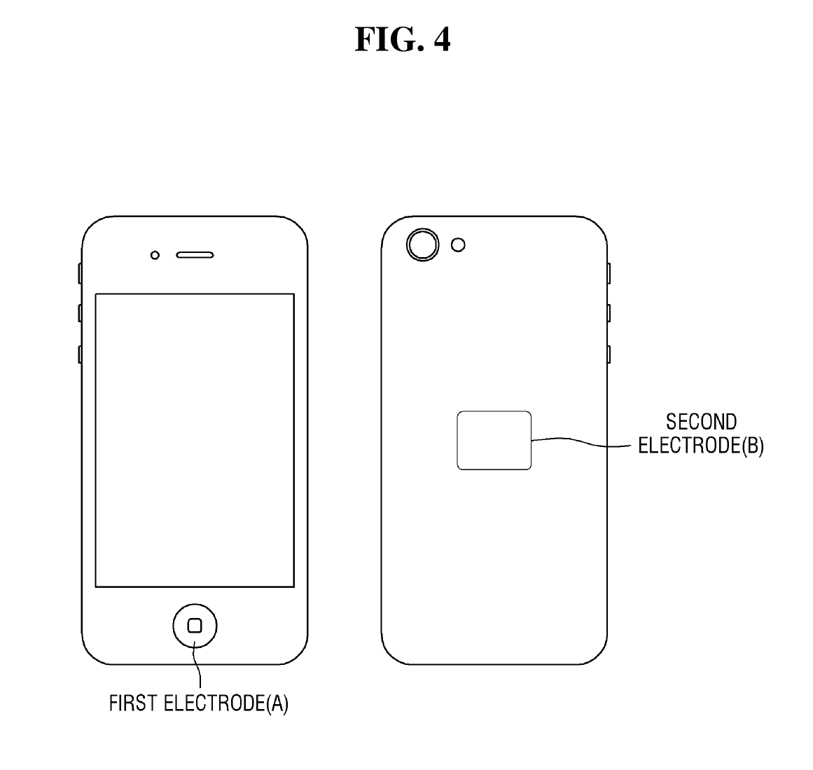

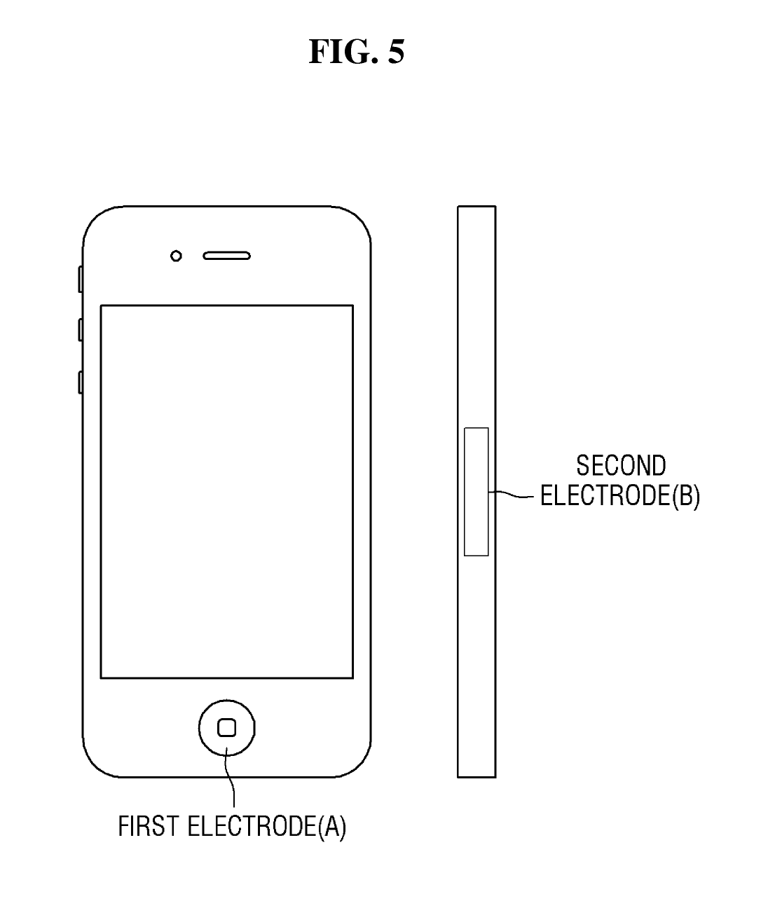

FIGS. 3 to 6 are diagrams illustrating a first example of the mobile terminal 200. Referring to FIGS. 3 to 6, the mobile terminal 200 may be configured to measure various biometric signals from a user with the use of the sensor 220 (e.g., an ECG sensor and a PPG sensor) provided in the main body 210. In the mobile terminal 200, a first electrode A is formed in a manipulation button (for example, a home button) provided in the main body 210, and a second electrode B is provided at a location apart from the manipulation button (or the home button) where the first electrode A is formed, for example, on the left side or the right side, or at the top, bottom or the rear, of the main body 210. The second electrode B may be formed at the rear of the main body 210, as illustrated in FIG. 4, or may be formed on a side of the main body 210, as illustrated in FIG. 5. The first and second electrodes A and B formed in the mobile terminal 200 may form an ECG sensor for measuring an ECG signal from the user.

For example, in response to the user placing a finger of one hand in contact with the first electrode A formed in the home button of the mobile terminal 200 and placing another body part (for example, a finger of the other hand) in contact with the second electrode B, an ECG signal may be measured from the user via the first and second electrodes A and B. In the first example of FIGS. 3 to 6, two electrodes formed apart from each other, i.e., the first and second electrodes A and B, may form an ECG sensor for measuring an ECG signal from the user. Alternatively, another electrode (i.e., a third electrode C) may be additionally provided at a location apart from the two electrodes so that the ECG sensor can be formed by three electrodes.

Alternatively, one or more electrodes may be additionally provided at location(s) apart from the three electrodes so that the ECG sensor may be formed by four or more electrodes. The mobile terminal 200 may include a PPG sensor (or an optical sensor) for measuring a PPG signal and/or a SpO₂ signal from the user. As discussed above, the PPG signal and the SpO₂ signal may be measured by irradiating light generated by a light-emitter (not illustrated) of the PPG sensor onto the tip of a finger or toe of the user and observing changes in the amount of light received by a light-receiver (not illustrated) of the PPG sensor after being transmitted through and/or reflected from the body of the user. The PPG sensor may be formed at a location where the electrodes of the ECG sensor are formed. In the first example of FIGS. 3 to 6, the PPG sensor that includes the light-emitter and the light-receiver may be formed at the manipulation button (or the home button) of the mobile terminal 200 together with the first electrode A of the ECG sensor.

The PPG sensor for measuring a PPG signal and/or a SpO₂ signal may include the light-emitter that has an R light-emitting diode (LED) generating R light having a wavelength of about 660 nm and an IR LED generating IR light and the light-receiver that has a photodiode and/or a phototransistor. According to the first example, various biometric signals such as ECG, PPG, and SpO₂ signals can be measured from the user in real time by using the sensor (e.g., the ECG sensor and the PPG sensor), and the blood pressure of the user can be estimated in real time by using the biometric signals. For the details of a method of measuring and analyzing biometric signals and a method of estimating blood pressure based on measured biometric signals, reference can be made to Korean Patent Application Nos. 2013-116158 and 2013-116165, the teachings of which are incorporated herein in their entirety.

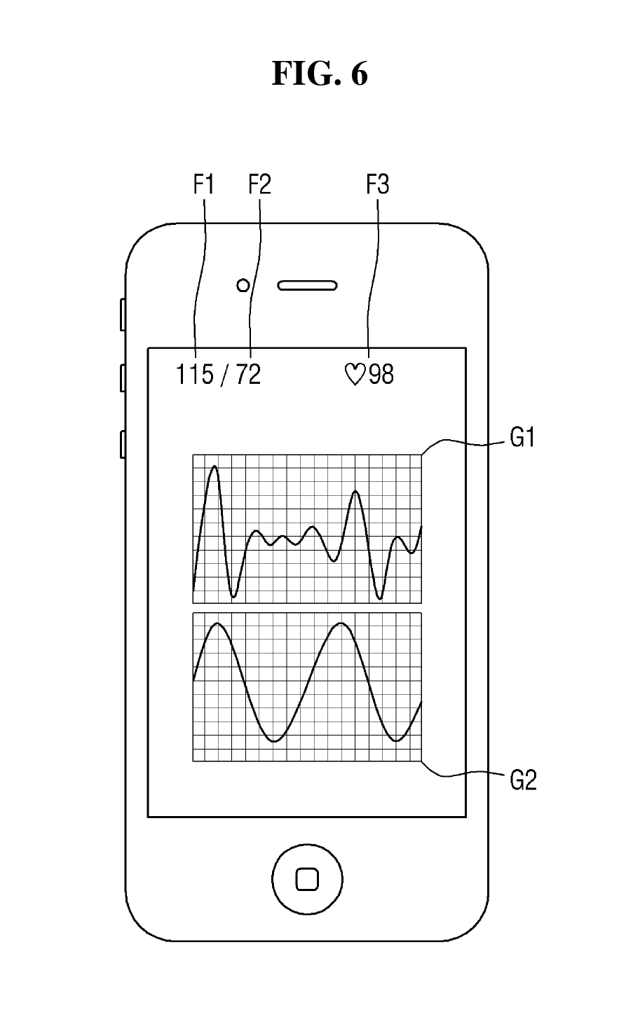

The mobile terminal 200 may be configured to display information regarding measured biometric signals and the results of analysis of the measured biometric signals on the display screen of the display 240 in the main body 210 and thus to deliver biometric signal information directly to the user. For example, numerical information such as systolic blood pressure F1, diastolic blood pressure F2 and pulse F3 or graph information showing changes in an ECG signal G1 or a PPG signal G2 may be displayed on the display screen of the display 240, as illustrated in FIG. 6. Also, a graph showing the user's real-time blood pressure information, which is estimated based on biometric signals, may be displayed on the display screen of the display 240. The content or style of information displayed on the display 240 may be selected/modified by the user.

### SECOND EXAMPLE OF MOBILE TERMINAL

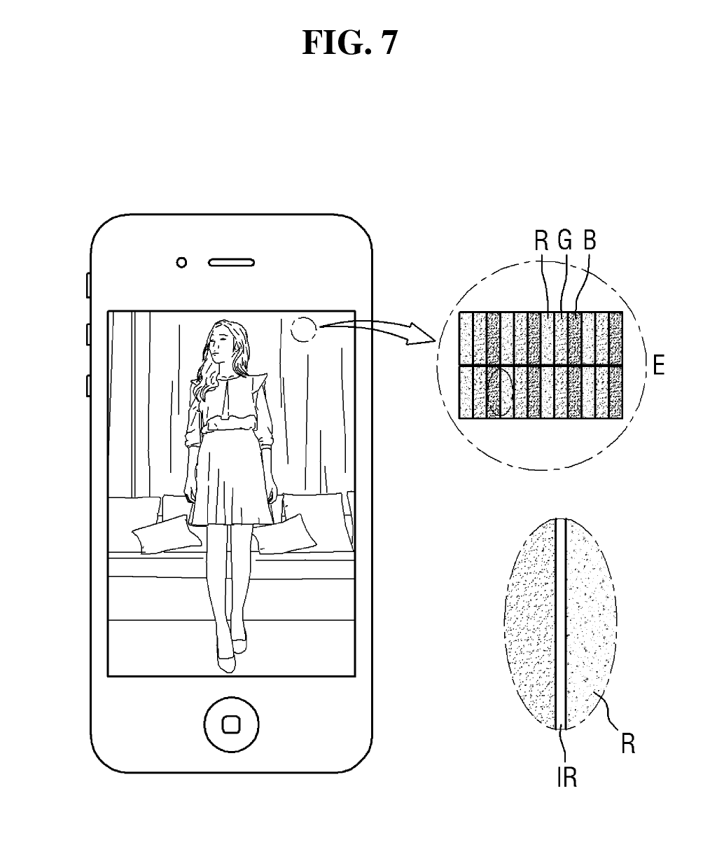

FIG. 7 is a diagram illustrating a second example of the mobile terminal 200. In the first example of FIGS. 3 to 6, the PPG sensor (or the optical sensor) having the light-emitter that irradiates light to the body of the user and the light-receiver that receives light transmitted through or reflected from the body of the user is needed to measure a PPG signal and/or a SpO₂ signal. Accordingly, the PPG sensor having the light-emitter and the light-receiver may be formed in the manipulation button (or the home button) of the mobile terminal 200 to measure a PPG signal and/or a SpO₂ signal from the user. On the other hand, in the second example of FIG. 7, no particular optical sensor may be provided. Instead, a PPG signal and/or a SpO₂ signal may be configured to be measured by using the display 240 provided in the mobile terminal 200.

More specifically, the display 240 of the mobile terminal 200 may include a measurement area E for measuring biometric signals (for example, a PPG signal and/or a SpO₂ signal) from the user. As discussed above, the irradiation of R light the human body is needed to measure a PPG signal, and the irradiation of R light and IR light to the human body is needed to measure a SpO₂ signal. For this, a pixel structure formed in the measurement area E of the display 240 may include IR sub-pixels in addition to R sub-pixels, green (G) sub-pixels and blue (B) sub-pixels, which generate R light, G light, and B light, respectively. In the second example, R light and IR light may be irradiated to the measurement area E due to the R sub-pixels and the IR sub-pixels in the measurement area E of the display 240. Accordingly, the functions of the light-emitter of the PPG sensor (or the optical sensor) for measuring a PPG signal and/or a SpO₂ signal may be performed.

The measurement area E of the display 240 may also include a light-receiver, which receives light reflected from the body of the user after being irradiated by the R sub-pixels and the IR sub-pixels. According to the second example, a PPG signal and/or a SpO₂ signal may be measured by using the display 240 without requiring an optical sensor in the mobile terminal 200. Although not specifically illustrated in FIG. 7, the mobile terminal 200, like its counterpart of FIGS. 3 to 6, may be configured to include a plurality of electrodes in the main body 210 to measure an ECG signal from the user.

### THIRD EXAMPLE OF MOBILE TERMINAL

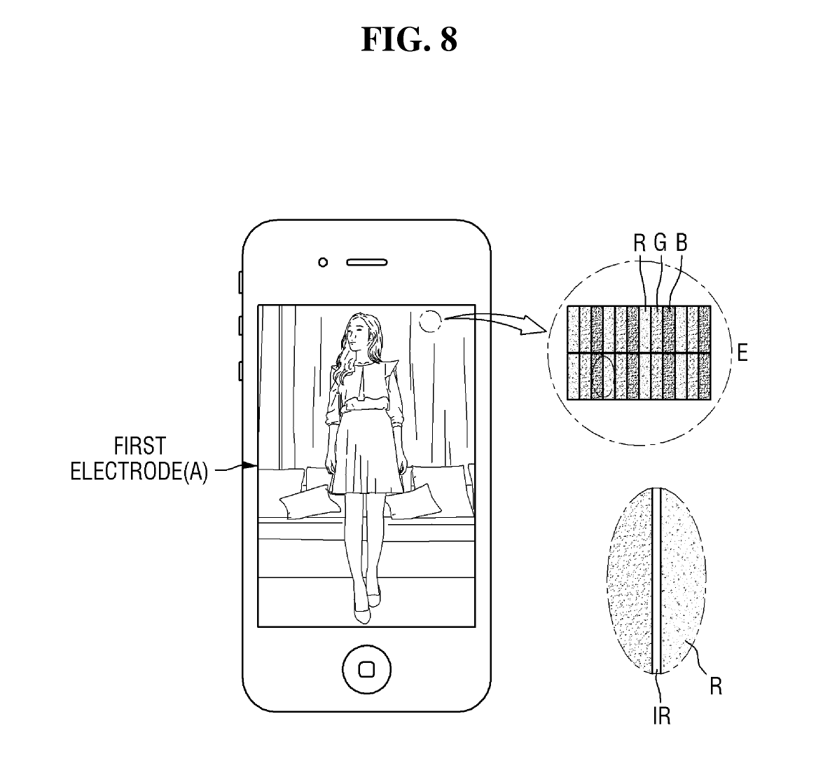

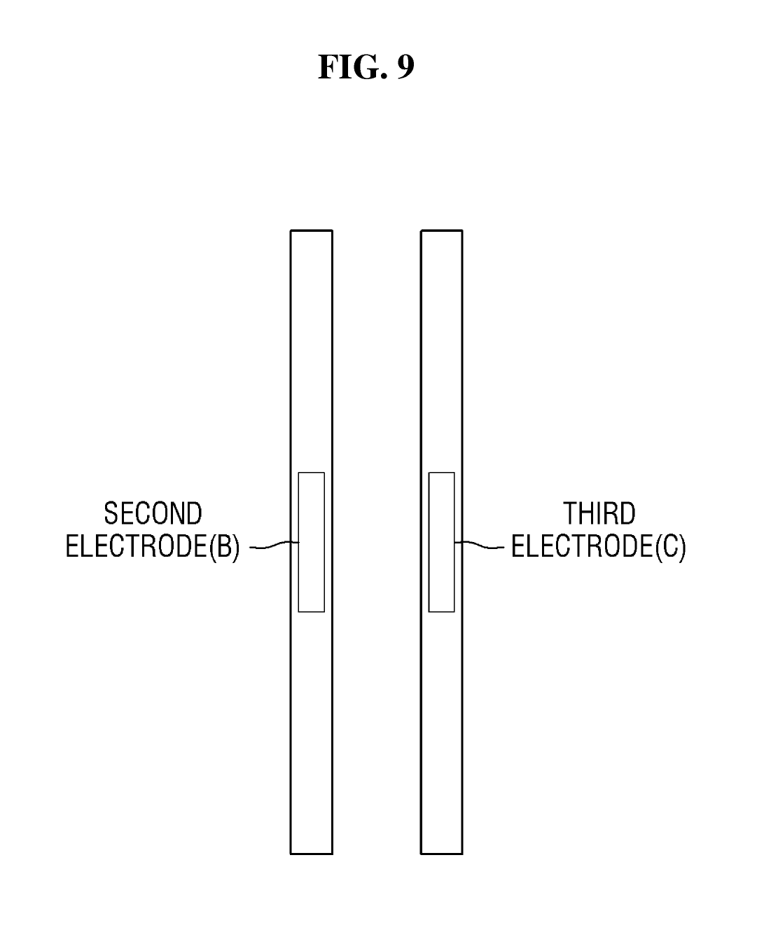

FIGS. 8 and 9 are diagrams illustrating a third example of the mobile terminal 200. One of a plurality of electrodes of an ECG sensor for measuring an ECG signal from the user may be disposed on the display screen of the display 240.

More specifically, in the third example shown in FIG. 8, a display screen of the display 240 of the mobile terminal may be used as a first electrode A to measure an electric signal of the human body, thereby measuring electrocardiogram (ECG). A second electrode B may be formed in a portion of the main body 210 where the display 240 is not provided. For example, in the third example of FIGS. 8 and 9, the second electrode B is formed on a side of the main body 210 (as seen in FIG. 9). Alternatively, the second electrode B may be provided in another part of the main body 210 (for example, at the top, bottom or the rear, or on the other side, of the main body 210). In the third example, a third electrode C for measuring an ECG signal may be provided at a location apart from the second electrode B. For example, as illustrated in FIG. 9, an electrode may be formed on the opposite side of the second electrode B as the third electrode C. Accordingly, the mobile terminal 200 may be configured to include three electrodes in the main body 210 to measure an ECG signal from the user with the use of an electric signal measured by the three electrodes. However, the third electrode C may not be provided. That is, similarly to FIG. 3, only the first and second electrodes A and B may be formed to measure an ECG signal. Alternatively, four or more electrodes may be formed apart from one another in the main body 210 to measure an ECG signal.

The third mobile terminal 200 may measure a PPG signal and/or a SpO₂ signal by using the display 240 in the same manner as its counterpart of FIG. 7. As described above, although the present disclosure has described specific matters such as specific components, the embodiments and the drawings are provided merely to assist in a general understanding of the present disclosure, and the present disclosure is not limited to the embodiments. Various modifications and changes can be made from the description by those skilled in the art.

Accordingly, the spirit and scope of the present disclosure should not be limited or determined by the above-described embodiments, and it should be noted that not only the claims which will be described below but also their equivalents fall within the spirit and scope of the present disclosure.

What is claimed is:

1. A mobile terminal having a function of measuring biometric signals of a user, comprising:
a main body; and
a display formed at a front of the main body and configured to display information to a user, wherein
the display includes a measurement area configured to measure biometric signals from the user,
a pixel structure formed in the measurement area of the display includes red (R) sub-pixels configured to generate R light, and infrared (IR) sub-pixels configured to generate IR light,
the measurement area of the display includes a light-receiver configured to receive light reflected from a body of the user, and
at least one of a photoplethysmogram (PPG) signal and an oxygen saturation (SpO₂) signal is measured through the measurement area.

2. The mobile terminal of claim 1, further comprising:
a first electrode disposed on a display screen of the display and a second electrode B formed in a portion of the main body where the display is not provided,
wherein at least one of an electrocardiogram (ECG) signal, the PPG signal, and the SpO₂ signal is measured through the first and second electrodes and the measurement area.

3. The mobile terminal of claim 2, wherein the second electrode is formed at one of a left side, a right side, the top, the bottom, and the rear of the main body.

4. The mobile terminal of claim 3, further comprising:
a third electrode provided at a location apart from the second electrode in the main body.

5. The mobile terminal of claim 1, further comprising:
a processor configured to analyze measured biometric signals,
wherein the processor is embedded in the main body.

6. The mobile terminal of claim 5, wherein the processor is configured to estimate blood pressure of the user in real time based on the measured biometric signals.

7. The mobile terminal of claim 2, wherein the display is configured to display at least one of information regarding measured biometric signals, results of analysis of the measured biometric signals, and the user's blood pressure estimated based on the measured biometric signals to the user.

8. A mobile terminal having a function of measuring biometric signals of a user, comprising:
a main body;
an ECG sensor including a first electrode formed in a home button provided in the main body, and a second electrode formed in a portion of the main body where the home button is not provided; and
a PPG sensor formed in a portion of the main body where the first or second electrode is formed and having a light-emitter configured to generate light to be irradiated to the body of the user, and a light-receiver configured to receive light reflected from the body of the user,
wherein at least one of an ECG signal, a PPG signal, and a SpO₂ signal is measured using at least one of the ECG sensor and the PPG sensor.

9. The mobile terminal of claim 8, wherein the PPG sensor is formed in the home button where the first electrode is formed.

10. The mobile terminal of claim 8, wherein the second electrode is formed at one of a left side, a right side, the top, the bottom and the rear of the main body.

11. The mobile terminal of claim 10, wherein the ECG sensor includes a third electrode provided at a location apart from the second electrode in the main body.

12. The mobile terminal of claim 8, further comprising:
a processor configured to analyze biometric signals measured by the ECG sensor or the PPG sensor,
wherein the processor is embedded in the main body.

13. The mobile terminal of claim 12, wherein the processor is configured to estimate blood pressure of the user in real time based on the biometric signals measured by the ECG sensor or the PPG sensor.

14. The mobile terminal of claim 8, further comprising:
a display configured to display at least one of information regarding biometric signals measured by the ECG sensor or the PPG sensor, results of analysis of the measured biometric signals, and the user's blood pressure estimated based on the measured biometric signals to the user,
wherein the display is embedded in the main body.

15. The mobile terminal of claim 8, wherein the light-emitter of the PPG sensor includes an R light-emitting diode (LED) configured to emit R light, and an IR LED configured to emit IR light, and the light-receiver of the PPG sensor includes at least one of a photodiode and a phototransistor.
